(19) United States
(12) Patent Application Publication
(10) Pub. No.: US 2019/0008552 A1
(43) Pub. Date: Jan. 10, 2019

(54) Medical Instruments With Multi-Faceted Edges

(71) Applicant: Bruce H. Levin, Oceanside, NY (US)

(72) Inventor: Bruce H. Levin, Oceanside, NY (US)

(21) Appl. No.: 16/028,381

(22) Filed: Jul. 5, 2018

Related U.S. Application Data
(60) Provisional application No. 62/528,845, filed on Jul. 5, 2017.

(51) Int. Cl. A61B 17/34

(52) U.S. Cl. CPC A61B 17/3417; A61B 2017/0073

(57) ABSTRACT

The present invention presents a medical instrument for penetrating cutaneous and sub-cutaneous tissue, causing less hemorrhage and patient pain. This medical device for insertion into tissue comprising one or more section, said section including a plurality of facets; and each facet is configured to deflect the tissue it contacts in a direction that is different than adjacent facets.

### RELATED APPLICATIONS

This application claims priority to U.S. Provisional Patent Application Ser. No. 62/528,845, filed Jul. 5, 2017, titled the same and incorporated herein as if set out in full.

### STATEMENT REGARDING FEDERALLY SPONSORED RESEARCH & DEVELOPMENT

Not applicable.

### INCORPORATION BY REFERENCE OF MATERIAL SUBMITTED ON A COMPACT DISC

Not applicable.

### BACKGROUND OF THE INVENTION

The present invention relates to medical instruments of special shapes, configurations, and geometries for penetrating or being positioned intravascularly, intraluminally, intracavity, or in cutaneous, subcutaneous, muscle, connective, organ, pericapsular, brain or other tissue, decreasing vascular trauma, irritation, inflammation, thrombogenicity, improving, air, gas or other fluid or vascular or luminal, intraluminal or other blood or fluid flow.

Conventional medical instruments penetrate or positioned in vascular, intraluminal or in cutaneous, sub-cutaneous, muscle, connective, organ or other tissue, resulting in well described tissue trauma or displacement, or intravascular effects on blood flow as the vector forces resulting from penetration, positioning or continued placement are in a primarily uniform direction with known tissue, resulting in increased focal damage, tissue or blood displacement intravascular, intraluminal or intracaitary hemorrhage and increased tissue or vascular pathology. The tissue force distributions resulting from typical needle insertion varied with distance and surface characteristics. Decreased tissue and vessel trauma and pain would ensue with more highly polished needle facet and other surfaces.

As an example, the following theory, which is not meant to be limiting, nor exclusionary is proposed. For analogic purposes, note that stealth fighter planes, submarines, or boats are less detectable by radar or sonar modalities in large part because their nonhomogenous surface geometries scatter reflected radar or sonar waves in multiple directions, thereby avoiding more uniform and focused return of radar or sonar signals back to the receiver. In effect, these energy waves are reflected in multiple pathways resulting in attenuation of energy back, in the direction of, and towards, the receiver. Hence, these reflected energy waves are attenuated, or defocused and essentially dispersed. It is well known that focused energy waves (or particles) can exert strong energetic forces at a given locus or loci as in the case of a laser, as opposed to nonfocused light energy. It is well understood what tissue displacement occurs when a bullet, arrow, needle, IV or other cannula, biopsy needle or scalpel penetrates skin, tissue or organs. Since the vector forces occur in a fairly predictable fashion depending upon geometry of the device tip, insertional direction, and tissue characteristics significant pathological effects on penetrated tissues are common.

By providing an entry tip or device of multifaceted and/or asymmetrical planes or curves, penetrating pathology and pain, hemorrhage can be decreased, regulated or adjusted. Further, it is well appreciated that intravascular or intraluminal catheters, stents, artificial valves, and other devices positioned intraluminally, for example intraarterially or intravenously, are subjected to the forces of fluid flow. In the case of a catheter or stent in a vein or artery, this flow is altered by the presence of the device and the directed forces intraluminally are reflected in such a way that vascular irritation, eddy currents, side flow with bacterial deposits or blood clots located proximal to the catheter tip are problematic. Further, simple catheter swinging can damage endovascular endothelial and other tissues. Further, vascular grafts often can occlude, or form thrombus. By making the surface of the catheter devices poly curved or polyfaceted or borrowing from the geometries of a stealth plane or boat catheter, stent or device related pathologies and damage will be reduced. Similarly, the same can be done for a vascular or other graft or stent on the intraluminal surface to decrease thrombogenicity, risk of large embolus, infection, and trauma at the suture sites. Further, fluid flow through IV or other tubing, monitoring devices, airway equipment would be improved by intraluminal nontraditional geometries as described. Therefore, nontraditional surface geometries internally, intraluminally, or on the surface of a multiplicity of devices results in defocusing and attenuation reflected vector forces and will be improve device related outcomes, minimize pathology, including tissue trauma, intravascular trauma, thrombosis and infection, and improve device longevity. Contrarily, by adjusting leading angle or curvature to have a more perpendicular orientation relating to the plane of travel, or including facet valleys, projectile lethality can be maximized.

### SUMMARY OF THE INVENTION

The present invention describes a medical instrument for penetrating cutaneous, sub-cutaneous and other tissues or for decreasing vascular trauma during placement or use of intravascular or other catheters, stents, tubes or other devices. An advantage of the present invention is to reduce the overall insertion trauma by configuring each facet to adjust to the tissue it contacts and disperse the force.

In other embodiments, the present invention provides a plurality planar facets or similar surfaces, ideally with the nonhomogenous arrangements and configurations. This surface geometry reflects or directs fluid in a less focused and more dispersive manner, and that tissue, fluid or other materials when subjected to compressive, non-compressive, penetrating, shearing or other forces along a given vector or vectors will be affected similarly when their reactive forces are generated against the geometrically fashioned surfaces.

### BRIEF DESCRIPTION OF THE DRAWINGS

FIG. 1 shows a front view of a first embodiment of the present invention.

FIG. 2 shows a front view of a second environment of the present invention.

FIG. 3 shows a front view of another embodiment of the present invention.

FIG. 4 shows a side view of an alternate embodiment of the present invention.

FIG. 5 shows a top view of the embodiments shown in FIG. 4.

FIG. 6 shows a top view of an alternate embodiment of the present invention.

FIG. 7 shows a top view of another embodiment of the present invention.

FIG. 8 shows a top view of another embodiment of the present invention.

FIG. 9 shows yet another top view of embodiment of the present invention.

FIG. 10 shows yet another top view of embodiment of the present invention.

FIG. 11 illustrates an interior or exterior surface of an embodiment of the present invention.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure or system. Further, the terms and phrases used herein are not intended to be limiting, but rather to provide an understandable description of the invention.

In the present invention, we claim that using higher numbers of poly planed facet or similar surfaces, ideally with the nonhomogenous arrangements and configurations found typically among stealth type low radar signal airplanes, boats or other vehicles or platforms will induce less focused tissue trauma, vascular injury, thrombogenesis, infection, pain, and less damaging alterations to blood flow. We hold this is because stealth surface geometry reflects or directs applied energy waves (i.e. radar waves) in a less focused and more dispersive manner, and that tissue, fluid or other materials when subjected to compressive, noncompressive, penetrating, shearing or other forces along a given vector or vectors will be affected in a similar manner when their reactive forces are generated against “stealth” geometrically fashioned surfaces. An energy wave is still an energy wave be it radar, light, sound, pressure or other mechanical form of energy.

Thrombosis is commonly attributed to the three principles of Virchow's Triad: endothelial trauma, changes in blood flow, and/or changes in blood coagulability or hypercoaguability 1. In an embodiment, increased with greater vessel diameter (4th power effect), laminar, fastest in the center according to Poiseuille's law of fluid flow in a closed tube (i.e. vein), more flow can be achieved with larger diameters. Applying Virchow's Triad for risk reduction a clinician should seek the most flow and the least disruption of flow as possible. Poiseuille's Law also accounts for velocity of vessel flow. Blood flow is slowest at the vein wall and fastest moving toward the center of the vein. Friction is created when fluid comes in contact with a stationary object like the vein wall, causing flow to become more sluggish at the vein wall. Passing a catheter into the vein creates more resistance to flow when the blood contacts the catheter surface. Turbulent low is erratic and usually should not be present except in very large veins related to high flow rates. Turbulence could also be created when laminar flowing blood contacts the catheter surface. Total catheter surface area impacts stasis and turbulence inside the vein. Damage of the vein wall starts a coagulation cascade that can be worsened with stasis and turbulent flow. Prior references indicate in simulated model, flow in a vessel could be reduced by 40%-93%, depending on the size of the catheter and vein used, or catheter-vein ratio. Catheter-vein ratio will result in less flow reduction as the vein increases in diameter moving proximally. A preventative thrombotic strategy would be to insert the smallest catheter necessary, in the largest vein possible, and in the most reasonably proximal zone. This should minimize catheter impact on vessel flow as it relates to Virchow's Triad.

In one embodiment, the present invention provides devices for influencing fluid flow around and/or inside a device, tube, lumen, vascular access, endotracheal tube, catheter, tubing or the like, and for influencing tissue damage resulting from the use of bullets, arrows, spears or other projectiles, as wells influencing the movement of said projectiles, missiles, torpedoes or also vessels, aircraft, spacecraft or drones, through viscous, aqueous, or solid or semisolid media or through varied atmospheric densities, or for providing more rapid and precise directionality or steering through fluids, deep sea, air, or atmospheric or sub-atmospheric conditions at usual or hypersonic speeds by raising, withdrawing, rotating or otherwise changing the orientation or degree of protrusion or intrusion of surface facets, planes, or curved surface components and this is made more effective with software and/or computer or artificial intelligence managed coordination of ailerons, flaps, rudders, elevators, thrust directors or the like, with or without real time human input.

In another embodiment, the present invention provides a medical device that is inserted into tissue. Unlike prior devices, the present invention has surface edges and sides that are multi-faceted, or multicurved. Each facet is configured to deflect the tissue it contacts in a direction that is different than adjacent facets. Thus, as the device is inserted into the tissue, there is no major tissue mass displaced or pushed in substantially the same direction or in a uniform direction by the instrument. Instead, the force exerted on the tissue is dispersed in a non-uniform manner and/or in a variety of angular or curvilinear directions. Alternatively, when each facet is configured to deflect the tissue it contacts, the insertion of the device creates a non-uniform insertion force, thereby reduce the force inserted upon one point of contact and avoid creating mass trauma to the point of contact. This reduces the overall insertion trauma since the force of insertion is not cumulative. Instead, it is dispersed which reduces the trauma to the tissue.

In another embodiment, the surface features of the present invention may be used at tip, body or trailing edge of any device that is intended to be inserted into a subject.

In yet another embodiment, the surface features may also repeat the length of the device, be located at predetermined location as well as on the interior external or both surfaces.

In another embodiment, the facets are arranged and configured to create canceling forces inside the tissue. This further reduces the insertion trauma. Insertion devices that may be used with the present invention include, but are not limited to, stents, scalpels, needles, catheters, trocars, valves and other medical devices that require insertion into tissue.

In other embodiments, the arrangement of facets or surface structure decreases focused vector forces decreasing tissue tearing and ripping. This further reduces the insertion trauma. Insertion devices that may be used with the present invention include, but are not limited to, stents, scalpels, needles, catheters, trocars, valves and other medical devices that require insertion into tissue.

Facets that may be used are comprised of planar sections that are varied in pattern, size, and angle as well as in other ways. In addition, the facets may vary in the angle of attack, height, depth, and spacing. The facets may also be concave, convex, or both, with primary or secondary or tertiary substructures.

In other embodiments, the outer and/or inner shapes and/or edges of the device are configured to influence the fluid flowing around the device or in the device in a controlled fashion. Shaping is a critical aspect of the design since the facets and aligned edges in the design direct fluid towards or away from the device depending on the application. Influencing fluid away or towards the device reduces wear, prevents bacteria adherence and has other effects.

To influence the fluid flow and flow pattern, or displacement of tissue, facet planes, multi planes, subplanes, curves, arcs, wavelike configurations, protuberances, vanes, or surface irregularities may be used. In addition, facets may be used that are comprised of planar sections that are varied in pattern, size, and angle as well as in other ways. In addition, the shape of the sections and/or edges may vary in the angle of attack, height, depth, and spacing to produce eddies in the fluid flow. The facets may also be concave, convex, or both. These structures may be configured to discourage flow stagnation (e.g. pooling, clotting, and clumping of the flow) that assists in preventing the adherence of bacteria.

In other embodiments, the present invention includes vortex generators on either the inner or outer surfaces. The vortex generators influence fluid flowing over the device, inside or through the device, or both and may alter fluid flow velocity, focus, distribution directionality, current type, and luminal fluid friction and or fluid surface friction and the like.

Other vortex generators that may be used with the invention include protrusions, indentations, grooves micro and or macro fractal grooves that penetrate into the device's surface or raised surfaces. Vortex generators may also include cylinders, ribs, plates, vanes, and airfoils that may be oriented parallel or normal to the flow or in any other desired location. In addition, the vortex generators may act to mix the fluid.

The present invention also provides a medical device that is inserted into a flow or fluid inside a mammal or another device. The medical device has sections that are each configured to influence the flow of fluid in many ways, including but not limited to, changing the direction of the flow, change the speed of the flow, or diverge the flow.

In one embodiment, each section contains one or more surface irregularities, including but not limited to, protuberances, indentations, ribs, grooves, cylinders, plates, vanes, airfoils. The sections may also be rough or smooth, or a combination of both in different locations to achieve the desired influence of the flow or fluid.

In another embodiment, the sections are planar but varied in multiple elements to achieve the desired influence of the flow or fluid, including but not limited to differences in pattern, size, angle and width, surface structure.

In another embodiment, the sections of the medical device can have surface irregularities, or planar surface, or a combination of both, or have both irregularities and planar surface on different area of the surface.

In another embodiment, the medical device can comprise any number of sections aligned in any combination, including but not limited to parallel, perpendicular, angular, top down, left right, front back to achieve the desired influence of the flow or fluid.

In another embodiments, the medical device can direct the flow or the fluid to any direction, including but not limited to, directing the fluid or flow towards or away from the interior of the medical device, towards or away the exterior of the medical device. The sections of the devices can be placed in combination to facilitate, fasten or to weaken, canceling each other's effect to achieve the desired influence of the flow or fluid.

In other embodiments, the sections are comprised of internal or external facets, protuberances, vanes, or surface irregularities configured to direct fluid towards or away from the device. The facets may have planar surfaces, accurate surfaces or combinations thereof.

The embodiments of the present invention may be used as needle, cannula, vascular access device, catheter, stent, drain, and endotracheal tube, valve, scalpel and tubing, a weapon, knife, bullet, spear, arrowhead, or projectile. In other embodiments the present invention provides at least one section, the section including a plurality of facets; and each facet is configured to deflect the tissue it contacts in a direction that is different than adjacent facets.

Accordingly, devices designed conforming to the concepts of the present invention may be used for devices implanted in a manner fluid flow in a body such as in the heart or other valve, be used as an anchoring device for heart or other valve or be used as a monitoring device.

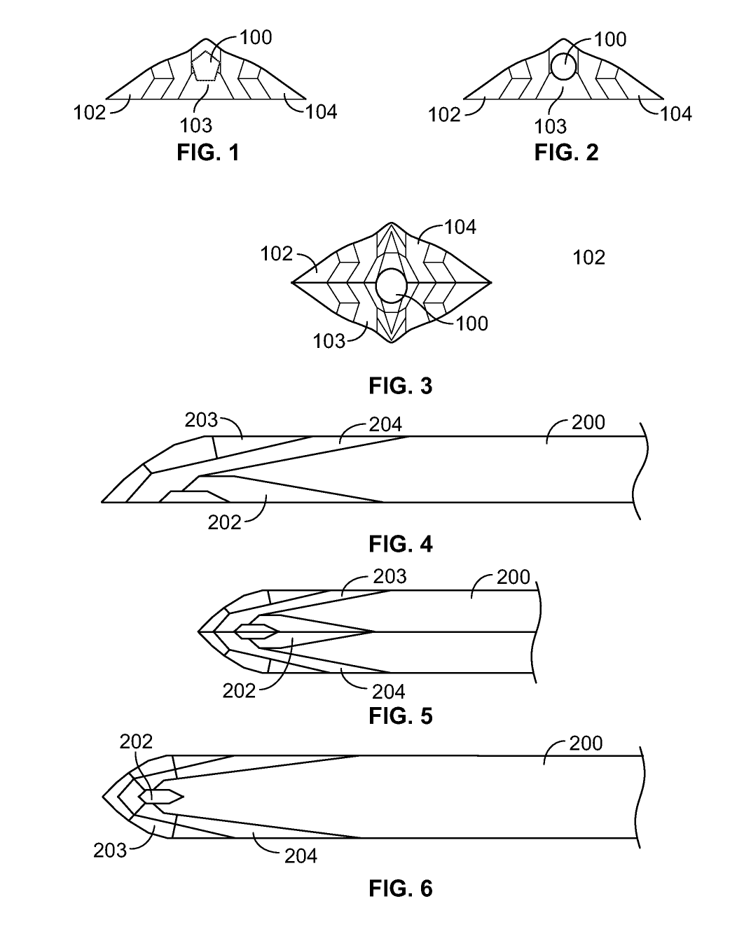

FIGS. 1-3 show front views of various embodiments of the present invention. Include is opening 100 as well as facets or surfaces 102-104 are arranged and configured as described above.

FIGS. 4 and 5 show an alternate embodiment of the present invention wherein facets or surfaces 202-204 are arranged and configured as described above. Surfaces 202-204 extend partially along the sides of device 200.

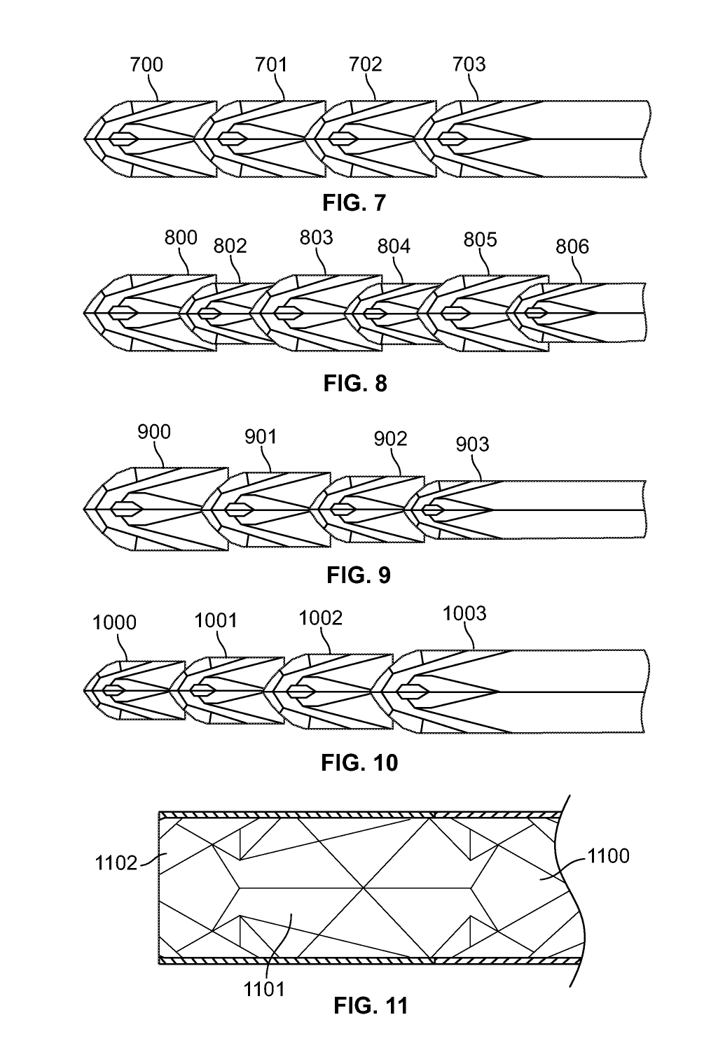

FIGS. 7-10 illustrate how the faceted surfaces may vary along the length of the device. FIG. 7 shows repeating facet patterns 700-703 which are similar or the same. FIG. 8 shows patterns 800-806 that alternate is size. FIG. 9 shows facets 900-903 that decrease in size from one end to another. FIG. 10 shows facets 1000-1103 that increase in size.

FIG. 11 shows how the exterior or interior surface of the device may be faceted with surfaces 1100-1102. The facets are arranged and configured as described above.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above described embodiments, methods, and examples, but by all embodiments and methods within the scope and spirit of the disclosure.

What is claimed is:

1. A device for insertion into tissue comprising:
at least one section, said section including a plurality of facets; and
each facet is configured to deflect the tissue it contacts in a direction that is different than adjacent facets.

2. The device of claim 1, wherein said facets is configured to adjust to fit the tissue surface it is in contact with.

3. The device of claim 1, wherein said facets reduce the overall insertion trauma on the tissue by creating a non-uniform insertion force.

4. The device of claim 1, wherein said facets create a noncumulative insertion force.

5. The device of claim 1, wherein said facets are arranged and configured to create canceling forces inside the tissue.

6. The device of claim 1, wherein said facets are arranged and configured to non-uniform angular insertion forces inside the tissue.

7. A device for influencing the flow of fluid inside a mammal or device comprising:
a plurality of sections; and
each section is configured to influence the flow of fluid with respect to the device.

8. The device of claim 7, wherein said sections are comprised of protuberances, vanes, or surface irregularities configured to direct fluid towards or away from the device.

9. The device of claim 7, wherein said sections are comprised of planar sections that are varied in pattern, size, and angle.

10. The device of claim 7. wherein said sections are comprised of protrusions, indentations, ribs, grooves, cylinders, ribs, plates, vanes, or airfoils that may be oriented parallel or normal to the flow or in any other desired location.

11. The device of claim 7, wherein said fluid is directed towards or away from the interior of the device.

12. The device of claim 7, wherein said fluid is directed towards or away from the exterior of the device.

13. The device of claim 7, wherein said fluid is directed towards or away from the device.

14. The device of claim 7, wherein said fluid is directed towards or away from the device in a plurality of different directions.

15. The device of claim 7, wherein said sections are comprised of internal or external facets, protuberances, vanes, or surface irregularities configured to direct fluid towards or away from the device.

16. The device of claim 1, wherein said facets have planar surfaces.

17. The device of claim 1, wherein said facets have accurate surfaces.

18. The device of claim 1 wherein said device is a needle, cannula, vascular access device, catheter, stent, drain, and endotracheal tube, valve, scapel and tubing,

19. The device of claim 7 wherein said device is a needle, cannula, vascular access device, catheter, stent, drain, and endotracheal tube, valve, scalpel and tubing.

20. The device of claim 1 wherein said device is a weapon, knife, bullet, spear, arrowhead, or projectile.

21. A device for insertion into a material comprising:
at least one section, said section including a plurality of facets; and
each facet is configured to deflect the tissue it contacts in a direction that is different than adjacent facets.
